(19) United States
(12) Patent Application Publication
(10) Pub. No.: US 2003/0013932 A1
(43) Pub. Date: Jan. 16, 2003

(54) Method for producing diarylmethane and its derivatives

(75) Inventor: Toshio Okuhara, Sapporo-shi (JP)

Correspondence Address: Mark J. Cohen, Scully, Scott, Murphy & Presser, 400 Garden City Plaza, Garden City, NY, 11530, US

(73) Assignee: Nippon Petrochemicals Co., Ltd., Tokyo (JP)

(21) Appl. No.: 10/163,668

(22) Filed: Jun. 6, 2002

(30) Foreign Application Priority Data
Jun. 20, 2001 (JP) 2001-186069

(51) Int. Cl. C07C 2/64

(52) U.S. Cl. 585/446

(57) ABSTRACT

The method for producing diarylmethane and its derivatives by condensation of an aromatic hydrocarbon and a methylenating agent is proposed, in which the aromatic hydrocarbon having at least one hydrogen atom that is directly connected to its benzene ring and a methylenating agent such as formalin are reacted in a specific temperature range in the presence of a heteropoly-acid of the condensation product of at least one oxide selected from the group of Mo, W, Nb and V with an oxyacid of P, Si, As or Ge.

### BACKGROUND OF THE INVENTION

1. Field of the Invention

The present invention relates to an improved method for producing diarylmethane and its derivatives which are useful as high boiling point aromatic solvents and chemical substances for various purposes. More particularly, the invention relates to a method for producing diarylmethane and its derivatives, using a methylenating agent such as formaldehyde.

2. Prior Art

Several methods have been proposed for producing diphenylmethanie by condensing benzene and formaldehyde in the presence of a catalyst such as sulfuric acid or other mineral acid and aluminum chloride.

For example, in Japanese Laid-Open Patent Publication No. 2-134332, the method to produce diarylmethane by the reaction of an aromatic compound such as toluene and formaldehyde in the presence of sulfuric acid. However, in the conventional method using a mineral acid such as sulfuric acid or aluminum chloride, several problems are cause to occur in the corrosion of reaction apparatus and the difficulty in the after-treatment of spent catalyst.

The handling of formaldehyde is difficult because it is gaseous substance at ordinary temperatures. So that, as the material to be fed to a reaction system, paraformaldehyde and trioxane are practically used, which are in solid state at ordinary temperatures.

Meanwhile, the formaldehyde is usually supplied by makers in the form of an aqueous solution of formaldehyde, the so-called formalin. Therefore, in industrial production, the above-mentioned paraformaldehyde and trioxane are often prepared from the above aqueous solution. Accordingly, if it is possible to use the formaldehyde in the form of an aqueous solution for the reaction with the foregoing aromatic hydrocarbon, it is quite advantageous to reduce the cost for formaldehyde in industrial practice because it is possible to use the inexpensive material supplied by makers of formaldehyde.

As the method to produce diarylmethanes using formalin, a reaction in the presence of sulfuric acid or aluminum chloride as a catalyst is known, as in the case using formaldehyde. However when these catalysts are used, it causes the problems such as the corrosion of apparatus and the difficulty in after-treatment of spent catalyst, as described above.

As the countermeasure to this problem, the reaction between phenol and formalin in the presence of solid acid catalysts such as zeolites is disclosed in Japanese Laid-Open Patent Publication No. 1-180835 and in U.S. Pat. No. 4,895,988. The reaction between phenol and formalin in the presence of heteropoly-acid is disclosed in Japanese Laid-Open Patent Publication No. 6-32756.

In the case of reaction of formalin and phenol compounds having high condensation activity with formaldehyde, the methods described in the forgoing patent publications are serviceable. However, in the reaction of formalin with aromatic hydrocarbons such as benzene having poor condensation activity with formaldehyde, the reaction is not always preferable because its yield is low.

Furthermore, disclosed in Japanese Laid-Open Patent Publication No. 6-87775 is the reaction between phenol and formalin using a heteropoly-acid catalyst that is carried out in the presence of aromatic compounds such as mesitylene. However, the obtainable product in this method is only bis(hydroxyphenyl)methane, while a condensation product of aromatic hydrocarbons with formaldehyde can not be produced.

Reaction between benzene and formalin in the presence of zeolite catalyst is disclosed in Japanese Laid-Open Patent Publication No. 10-231259, however, it involves a problem that the reaction must be carried out at a high temperature and a high pressure.

### BRIEF SUMMARY OF THE INVENTION

The object of the present invention is to provide a method for producing diarylmethane and its derivatives through the condensation of aromatic hydrocarbons and methylenating agent in the presence of heteropoly-acid catalyst, which method can be employed without difficulty under mild reaction conditions in industrial production sites.

A first aspect of the present invention, therefore, relates to a method for producing diarylmethane and its derivatives that are represented by the following general formula I. The method is characterized by the reaction of an aromatic hydrocarbon with a methylenating agent in the presence of a heteropoly-acid.

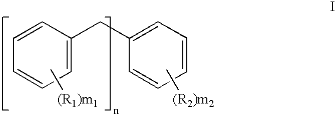

wherein each of m₁ and m₂ is an integer from 0 to 3, n is 1 or 2, and each of R₁ and R₂ is an alkyl group having 1 to 4 carbon atoms, respectively.

A second aspect of the present invention relates to the method for producing diarylmethane and its derivatives in the first aspect, wherein the above aromatic hydrocarbon is benzene.

A third aspect of the present invention relates to the method for producing diarylmethane and its derivatives in the first or second aspect, wherein the above methylenating agent is formalin.

A fourth aspect of the present invention relates to the method for producing diarylmethane and its derivatives in the first through third aspects, wherein the above reaction is carried out at a temperature in the range of 130 to 300° C. and under an elevated pressure that is higher than the autogenous pressure of the reaction system.

A fifth aspect of the present invention relates to the method for producing diarylmethane and its derivatives as described in the first through fourth aspects, wherein the obtained reaction mixture is separated into an aqueous phase and an oily phase, and the heteropoly-acid recovered from the aqueous phase is recycled into the reaction of aromatic hydrocarbon with methylenating agent.

### DETAILED DESCRIPTION OF THE INVENTION

In the following, the present invention will be described in more detail.

The aromatic hydrocarbons used in the present invention are those having at least one hydrogen atom that is directly connected to a benzene ring such as benzene and alkylbenzenes such as toluene, xylene, ethylbenzene, cumene, trimethylbenzene, ethyltoluene, and diethylbenzene. As it will be understood in view of the above exemplified compounds, the benzene ring can have lower alkyl substituent groups such as methyl, ethyl, propyl or butyl group besides the hydrogen atom. The alkyl substituent groups can be single or plural. Furthermore, the plurality of alkyl groups may be the same ones or different ones. In the present invention, one or a mixture of two or more kinds of the aromatic hydrocarbons can be used.

The above-mentioned methylenating agent to be used in the method of the present invention is not limited as far as the compound can be condensed with aromatic hydrocarbons such as formalin, formaldehyde, paraform-aldehyde, trioxan and methylal. Formalin is preferable in view of its low cost and easiness in handling.

The formalin used in the method of the present invention is an aqueous solution of formaldehyde, which can contain methanol as a stabilizing agent. The formalin is commonly supplied as an aqueous solution of formaldehyde of about 40% in concentration. The concentration is not restricted to such a value but it is possible to use formalin in lower concentration. However, the concentration of formaldehyde (including its dimer and higher polymers) in formalin is preferably more than 20% by weight. Although the upper value of formaldehyde concentration is not limited as far as the formalin is in the state of an aqueous solution, the concentration is generally lower than 80% by weight. The formaldehyde exists generally in a hydrated form in the aqueous solution.

The molar ratio of the aromatic hydrocarbon to the methylenating agent as represented by (aromatic hydrocarbon)/(methylenating agent) is in the range of 1 to 100, preferably 5 to 40. By adjusting the molar ratio, the ratio of the of compounds having two benzene rings to the compounds having three benzene rings in the product can be controlled.

The heteropoly-acid used in the method of the present invention is an acid produced by the condensing at least one member of oxide selected from the group of Mo, W, Nb and V, with oxyacid of P, Si, As or Ge. The heteropoly-acids are exemplified by phosphomolybdic acid, phosphotungstic acid, phosphomolybdotungstic acid, phosphomolybdovanadic acid, phospho-tungstovanadic acid, phosphomolybdoniobic acid, silicotungstic acid, silico-molybdic acid, silicomolybdotungstic acid, silicomolybdotungstovanadic acid, germannotungstic acid, arsenomolybdic acid and arsenotungstic acid.

The reaction temperature can be arbitrarily selected within the range of 130 to 300° C. The reaction temperature below 130° C. is not preferable because the rate of reaction is low and a by-product of oxygen-containing compound such as benzyl alcohol is formed in the use of some kind of catalyst. On the other hand, if the reaction temperature is higher than 300° C., it is not preferable because side reaction such as the transferring of alkyl groups is caused to occur. The reaction temperature below 200° C. is more preferable.

The pressure of reaction is not especially limited, while it is preferable that the reaction is carried out under a pressure above an autogenous pressure.

After the reaction, the obtained reaction mixture is subjected to oil/water separation process to separate the mixture into an oily phase and an aqueous phase. Then, the diarylmethane and its derivatives are recovered from the oily phase through a known separating method such as distillation. The recovered aqueous phase containing heteropoly-acid can be recycled to the reaction system. In this step, the separated aqueous phase can be used intact or, if desired, it is concentrated for the reuse.

In the following, the present invention will be described in more detailed with reference to several examples.

### EXAMPLE 1

To a 100 ml autoclave with a stirrer were added 40 ml of commercially available benzene, 6.72 ml of formalin having a concentration of 37% by mass (90 mmol as formaldehyde) and 4.2 mmol of phosphotungstic acid. The contents were stirred at 160° C. for 2 hours.

After the reaction, the yields of diphenylmethane (DPM) and dibenzylbenzene (DBB) were determined by gas chromatographic (GC) analysis on the oily phase.

The yields according to the quantity of used formaldehyde are shown in the following Table 1.

TABLE 1

| | | Yield | Yield | |
|---|---|---|---|---|
| Example | Catalyst | of DPM | of DBB | Total Yield |
| Example 1 | H₃PW₁₂O₄₀ | 36.3 | 11.1 | 47.4 |
| Example 2 | H₄SiW₁₂O₄₀ | 27.3 | 9.6 | 36.9 |
| Example 3 | H₃PMo₁₂O₄₀ | 23.5 | 14.3 | 37.8 |
| Comparative | p-toluene- | 4.1 | 1.1 | 5.2 |
| Example 1 | sulfonic acid | | | |
| Comparative | H₃PO₄ | 0.0 | 0.0 | 0.0 |
| Example 2 | | | | |

### EXAMPLE 2

In place of phosphotungstic acid, 4.2 mmol of silicotungstic acid was used and the reaction was carried out in the like manner as in Example 1. The yields of the products are shown also in Table 1.

### EXAMPLE 3

In place of phosphotungstic acid, 4.2 mmol of phosphomolybdic acid was used and the reaction was carried out in the like manner as in Example 1. The yields of the products are shown in Table 1.

### Comparative Example 1

In place of phosphotungstic acid, 12.6 mmol of p-toluene sulfonic acid was used and the reaction was carried out in the like manner as in Example 1. The yields of the products are shown in Table 1.

### Comparative Example 2

In place of phosphotungstic acid, 4.2 mmol of phosphoric acid was used and the reaction was carried out in the like manner as in Example 1. The yields of the products are shown in Table 1.

### ADVANTAGES OF THE INVENTION

By employing the method of the present invention, diarylmethane and its derivatives can be produced by using aromatic hydrocarbons and methylenating agent in the presence of industrially available catalysts under moderate reaction conditions.

What is claimed is:

1. A method for producing diarylmethane and its derivatives, which are represented by the following general formula I, comprising reacting an aromatic hydrocarbon with a methylenating agent in the presence of a heteropoly-acid, 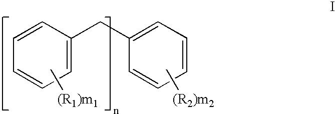
wherein each of m₁ and m₂ is an integer from 0 to 3 independently, n is 1 or 2, and each of R₁ and R₂ is an alkyl group having 1 to 4 carbon atoms.

2. The method for producing diarylmethane and its derivatives as claimed in claim 1, wherein said aromatic hydrocarbon is benzene.

3. The method for producing diarylmethane and its derivatives as claimed in claim 1 or 2, wherein said methylenating agent is formalin.

4. The method for producing diarylmethane and its derivatives as claimed in any one of claims 1 to 3, wherein said reaction is carried out at a temperature in the range of 130 to 300° C. under an elevated pressure which is higher than an autogenous pressure of the reaction system.

5. The method for producing diarylmethane and its derivatives as claimed in any one of claims 1 to 4, wherein the obtained reaction mixture is subjected to oil/water separation process and heteropoly-acid contained in the obtained in aqueous phase is recycled to the reaction of aromatic hydrocarbon with methylenating agent.
